(19) United States
(12) Patent Application Publication
(10) Pub. No.: US 2022/0347444 A1
(43) Pub. Date: Nov. 3, 2022

(54) APPARATUS AND METHOD FOR DRAINING CYSTIC FLUIDS FROM JOINTS

(71) Applicants: Joseph R. O'Brien, McLean, VA (US); Babak Arvanaghi, McLean, VA (US)

(72) Inventors: Joseph R. O'Brien, McLean, VA (US); Babak Arvanaghi, McLean, VA (US)

(21) Appl. No.: 17/302,287

(22) Filed: Apr. 29, 2021

(51) Int. Cl. A61M 27/00

(52) U.S. Cl. CPC A61M 27/00; A61M 2210/1003

(57) ABSTRACT

A method and apparatus for treating a synovial cyst is disclosed. The method includes determining a presence and location of a cyst within a body, penetrating the cyst through a penetration bore to release a synovial fluid accumulation from within the cyst. A suction is applied to the cyst to via the penetration bore to extract a volume of the fluid accumulation from within the cyst. A weeping tube is inserted in the penetration bore to communicate synovial fluid from the cyst to maintain the cyst in a depressed condition for a predetermined temporal period. The weeping tube an external sidewall and an interior channel configured to communicate a synovial fluid from the synovial cyst. A plurality of protrusions about the external sidewall engage with a body tissue surrounding a penetration bore in the body tissue to retain the weeping tube at a desired placement within the penetration bore.

### BACKGROUND OF THE INVENTION

The present invention relates to cystic fluid accumulation in joints of the human body, and more particularly to apparatus and methods for ameliorating cystic fluid accumulation.

Cyst growth in the human body often cause pain and disability. For instance, in the lumbar spine facet cysts pinch nerves and cause sciatica. Popliteal cysts cause Baker's cysts and cause knee pain. Most cysts require surgery to provide the patient relief.

While some cysts may be drained by emplacement of a needle, recurrence of the cyst growth is common with simple drainage techniques.

Presently, there are no systems available to drain cysts in the human body continuously for a prolonged period.

As can be seen, there is a need for relieving accumulation of synovial fluid from a cyst and to decompress the cyst over a prolonged period of time.

### SUMMARY OF THE INVENTION

In one aspect of the present invention, a method of treating a cyst is disclosed. The method includes determining a presence and a location of the cyst within a body. The cyst is penetrated through a penetration bore to release a fluid accumulation from within the cyst. The cyst is suctioned via the penetration bore to extract a volume of the fluid accumulation from within the cyst. A weeping tube is then inserted in the penetration bore. The weeping tube has an interior channel configured to communicate a residual fluid volume from the cyst to maintain the cyst in a depressed condition for a predetermined temporal period.

In some embodiments, the weeping tube includes an elongate cylindrical body having an external sidewall with the interior channel extending therethrough. A plurality of protrusions are disposed in a spaced apart relation about the external sidewall. The plurality of protrusions are configured to engage with a body tissue surrounding the penetration bore to retain the weeping tube at a desired placement within the penetration bore.

In some embodiments, the method also includes providing a Fluoroscopic Guidance while inserting the weeping tube in the penetration bore.

In a preferred embodiment, the weeping tube is formed of a biodegradable material. The biodegradable material is selected to communicate the residual fluid volume for the predetermined temporal period. The predetermined temporal period is between about three to six months.

In other aspects of the invention, a surgical implant for treatment of a synovial cyst is disclosed. The surgical implant includes a weeping tube having an external sidewall and an interior channel configured to communicate a synovial fluid from the synovial cyst to maintain the synovial cyst in a depressed condition for a predetermined temporal period. A plurality of protrusions are disposed in a spaced apart relation about the external sidewall. The plurality of protrusions are configured to engage with a body tissue surrounding a penetration bore in the body tissue to retain the weeping tube at a desired placement within the penetration bore.

In some embodiments, the weeping tube is formed of a human implantable material. The human implantable material is a biodegradable material selected to communicate the synovial fluid for the predetermined temporal period. The predetermined temporal period is between about three to six months.

These and other features, aspects and advantages of the present invention will become better understood with reference to the following drawings, description and claims.

### BRIEF DESCRIPTION OF THE DRAWINGS

FIG. 1 is a side view highlighting the spinal column showing the presence of a lumbar spine facet cyst.

FIG. 2 is a cross section of the spinal joint showing a facet cyst in an initial assessment and preparation step of the method.

FIG. 3 is a cross section of a cyst penetration step of the method.

FIG. 4 is a cross section of a cyst evacuation step removing the synovial fluids from the cyst.

FIG. 5 is a cross section of a drainage tube implantation step in the cyst.

FIG. 6 is a perspective view of biodegradable cyst drainage tube.

### DETAILED DESCRIPTION

The following detailed description is of the best currently contemplated modes of carrying out exemplary embodiments of the invention. The description is not to be taken in a limiting sense but is made merely for the purpose of illustrating the general principles of the invention.

Broadly, embodiments of the present invention provide and apparatus and method for removing the accumulation of synovial fluid from a cyst and maintaining decompression of the cyst over a prolonged temporal period. The method entails the implantation of a biodegradable tube into a cyst to allow drainage of the synovial fluid from the cyst over a prolonged temporal period to prevent the reemergence of the cyst.

The apparatus and method of the present invention will be described in the context of a lumbar spine facet cyst. The same may be applied for the amelioration of other joint cysts in the body.

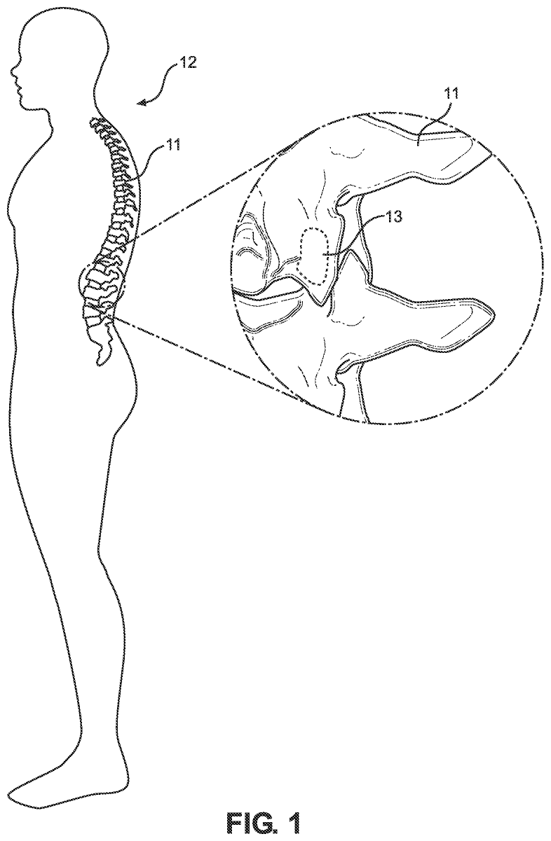

As seen in reference to FIG. 1, a patient 12 suffering from a spinal column 11 facet cyst 13 is shown. The facet cyst 13 develops with the accumulation of synovial fluid becoming entrapped by the synovial lining of the joint. The presence of the cyst may be identified through X-ray and other imaging techniques.

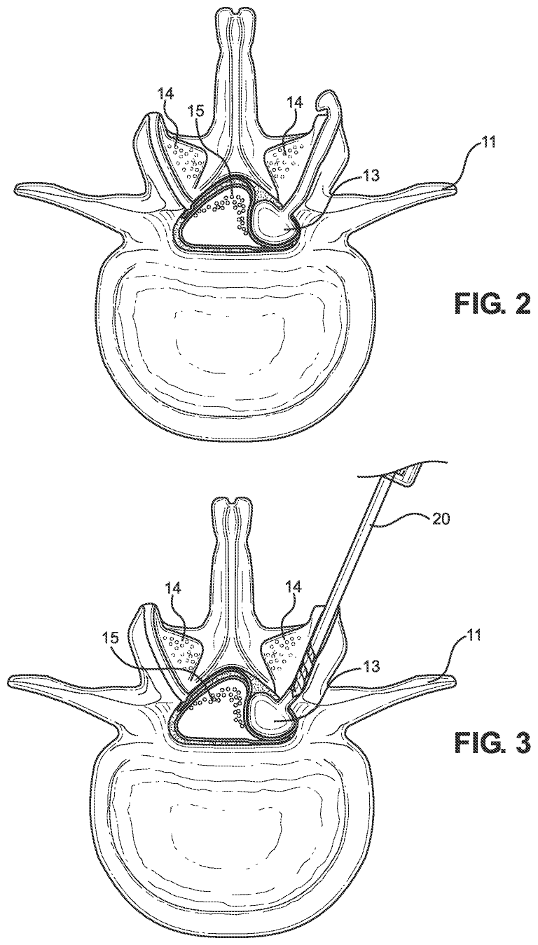

A method of treating a cyst 13 according to aspects of the invention is to identify the location and orientation of the cyst 13, in the facet joint 14. As shown in reference to FIG. 2, the cyst 13 is causing an undesired compression of the spinal nerves 15 carried within the vertebra 11.

Once identified, the cyst 13 is penetrated, as shown in reference to FIG. 3, such as with a rotary boring bit 20 or a large gauge needle. Fluoroscopic Guidance techniques may be employed to properly guide the penetration of the cyst 13 and through subsequent steps of the procedure.

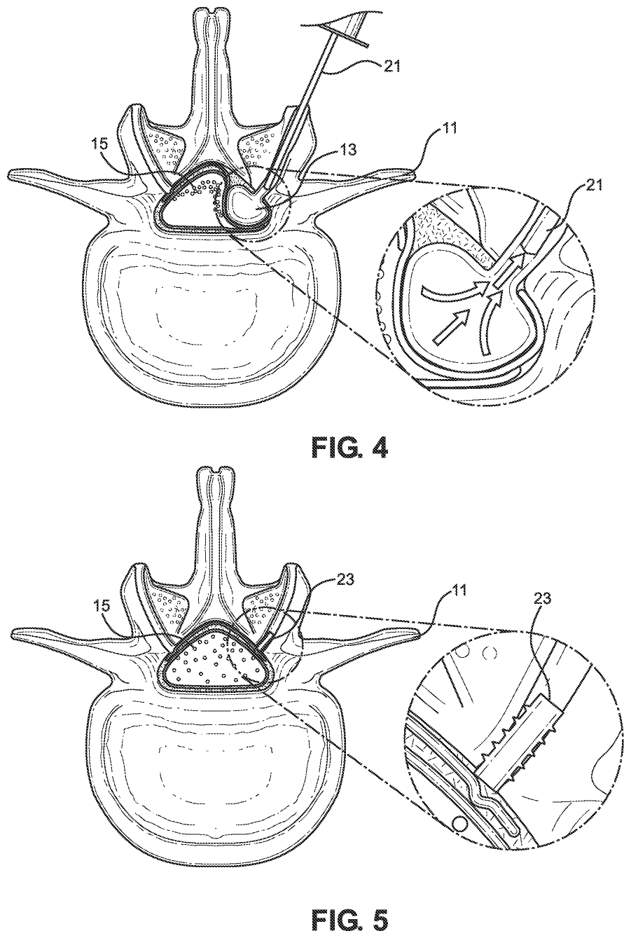

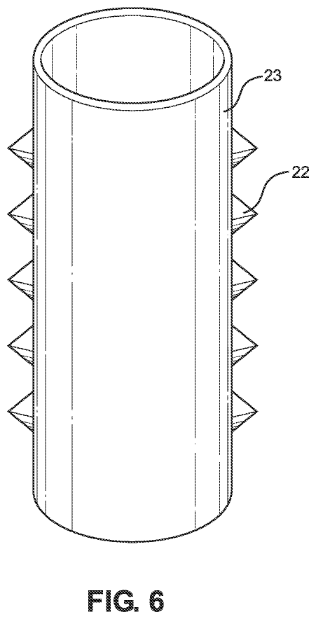

Once penetrated, the synovial fluid within the cyst 13 will be released due to the fluid pressure within the cyst 13. To extract residual synovial fluid from the cyst 13, the synovial fluid may be evacuated from the cyst 13 by a needle 21 operatively connected to a vacuum source, as shown in reference to FIG. 4. The vacuum source may be a manually operated syringe or other source of vacuum, such as a pump.

Once the cyst 13 has been fully evacuated, a weeping tube 23 may be inserted at the penetration point of the cyst 13. The weeping tube 23 is formed as an elongate cylindrical tube having an external sidewall and an interior channel extending therethrough. The plurality of protrusions 22 are disposed in a spaced apart relation about an outer wall of the weeping tube 23. The plurality of protrusions 22 are configured to engage with the body tissues within the penetration bore. The weeping tube 23 allows additional synovial fluids to seep from the penetration bore over an extended temporal period. The additional synovial fluids may emerge in the surrounding tissues as the patient resumes normal body movements following the procedure.

The biodegradable material is selected from materials suitable for implantation, such as lactic acid based implantable materials. The biodegradable material should be selected to have a longevity of roughly three to six months to allow the cyst 13 to be maintained in a decompressed condition to allow recovery of the synovial membrane and prevent the reemergence of the cyst.

A length, an outer diameter of the weeping tube, and an inner diameter of the channel may be selected based on the locus of the cyst in the patient's body, the size of the cyst, and anthropomorphic measurements of the patient. Examples include synovial cysts of the facet joints, ganglion cysts of the wrist, and popliteal cysts of the knee. By way of non-limiting example, fora lumbar facet cyst, the length may be 0.5 cm to 2.0 cm.

It should be understood, of course, that the foregoing relates to exemplary embodiments of the invention and that modifications may be made without departing from the spirit and scope of the invention as set forth in the following claims.

What is claimed is:

1. A method of treating a cyst, comprising:
determining a presence and location of the cyst within a body;
penetrating the cyst through a penetration bore to release a fluid accumulation from within the cyst;
suctioning the cyst to via the penetration bore to extract a volume of the fluid accumulation from within the cyst; and
inserting a weeping tube in the penetration bore, the weeping tube having an interior channel configured to communicate a residual fluid volume from the cyst to maintain the cyst in a depressed condition for a predetermined temporal period.

2. The method of claim 1, wherein the weeping tube further comprises:
an elongate cylindrical body having an external sidewall with the interior channel extending therethrough; and
a plurality of protrusions disposed in a spaced apart relation about the external sidewall, the plurality of protrusions configured to engage with a body tissue surrounding the penetration bore to retain the weeping tube at a desired placement within the penetration bore.

3. The method of claim 1, further comprising:
providing Fluoroscopic Guidance while inserting the weeping tube in the penetration bore.

4. The method of claim 1, wherein the weeping tube is formed of a biodegradable material.

5. The method of claim 4, wherein the biodegradable material is selected to communicate the residual fluid volume for the predetermined temporal period.

6. The method of claim 5, wherein the predetermined temporal period is between about three to six months.

7. A surgical implant for treatment of a synovial cyst, comprising:
a weeping tube having an external sidewall and an interior channel configured to communicate a synovial fluid from the synovial cyst to maintain the synovial cyst in a depressed condition for a predetermined temporal period; and
a plurality of protrusions disposed in a spaced apart relation about the external sidewall, the plurality of protrusions configured to engage with a body tissue surrounding a penetration bore in the body tissue to retain the weeping tube at a desired placement within the penetration bore.

8. The surgical implant of claim 7, wherein the weeping tube is formed of a human implantable material.

9. The surgical implant of claim 8, wherein the human implantable material is a biodegradable material selected to communicate the synovial fluid for the predetermined temporal period.

10. The surgical implant of claim 9, wherein the predetermined temporal period is between about three to six months.
